(19) United States
(12) Patent Application Publication
(10) Pub. No.: US 2005/0203207 A1
(43) Pub. Date: Sep. 15, 2005

(54) Dental compositions and kits containing bitterness inhibitors, and related methods

(76) Inventors: Thomas Klettke, Diessen (DE); Thomas Luchterhandt, Greifenberg (DE); Sumita B. Mitra, West St. Paul, MN (US); Armineh Khachatoorian, La Crescenta, CA (US); Josef Silberg, Starnberg (DE)

Correspondence Address: 3M INNOVATIVE PROPERTIES COMPANY, PO BOX 33427, ST. PAUL, MN, 55133-3427, US

(21) Appl. No.: 11/041,114

(22) Filed: Jan. 21, 2005

Related U.S. Application Data
(60) Provisional application No. 60/538,024, filed on Jan. 21, 2004.

(51) Int. Cl. C08K 3/00; A61F 2/00

(52) U.S. Cl. 523/115; 523/118

(57) ABSTRACT

The invention relates to dental compositions, such as dental or orthodontic adhesives, dental or orthodontic cements, or impression materials, and/or kits that contain a bitterness inhibitor. The dental compositions and kits are useful for blocking the perception of bitterness caused by a bitter tastant.

This application claims the benefit of U.S. Provisional Application No. 60/538,024, filed Jan. 21, 2004, which is incorporated herein by reference in its entirety.

### FIELD OF INVENTION

The present invention relates to dental compositions (e.g., impression materials and dental cements) and kits that contain bitterness inhibitors that block bitter taste receptors.

### BACKGROUND

The sensory perception of taste typically involves the interaction of a taste-active molecule, commonly referred to as a tastant, with taste receptor cells that reside in the papillae of the tongue and are the end organs of taste. Each taste modality affects receptor cells through distinct mechanisms. The taste buds then relay the information to the central nervous system.

Dental compositions, such as those used by dental practitioners for a variety of oral treatments, often consist of active chemical ingredients (e.g., catalyst components) that impart a bitter taste to the composition. Examples of such bitter-tasting compositions include, for example, certain cement compositions (e.g. orthodontic band cements) and certain polyether impression materials. When the patient's tongue comes into contact with these materials, for example during placement in the mouth or during its use period, the patient may experience an unpleasant, bitter taste.

One approach for eliminating the bitter taste of some dental compositions (e.g., certain orthodontic cements and certain impression materials) has been the addition of flavoring agents in attempts to mask the bitter taste of the compositions. However, this approach has typically not resulted in satisfactorily masking the overall unpleasant bitter taste perception experienced with such compositions.

Another approach for eliminating the bitter taste of some dental compositions (e.g., certain hardenable compositions) has been to use alternative components (e.g., catalyst components) in attempts to eliminate components that may be causing the bitter taste. For example, alternative catalyst components have been discussed, but such alternative catalyst components are typically thought to have undesirable properties (e.g., acidity), which can lead to, for example, corrosion problems (e.g., corrosion of the packaging system).

Additional approaches for eliminating the bitter taste of certain dental compositions are needed.

### SUMMARY

Conventional techniques for reducing the perception of bitterness in dental compositions, such as adding flavoring agents to the composition, typically are not able to satisfactorily mask the unpleasant bitter taste of certain ingredients present in these compositions. Accordingly, in one aspect, the present invention features a dental composition that includes a reactive component and a bitter tastant in combination with a bitterness inhibitor. Typically the reactive component includes a hardenable or polymerizable component. The dental composition may be, for example, a dental adhesive, an orthodontic adhesive, a dental cement, an orthodontic cement, a filling material, or an impression material such as a polyether impression material. Suitable bitterness inhibitors include nucleotides, for example, which are preferably monophosphates, such as adenosine monophosphate.

In another aspect, the present invention provides a method of inhibiting a bitter taste of a reactive dental composition that results from contacting a taste tissue of a subject with a bitter tastant. The method includes administering to the subject a dental composition that includes a reactive component, a bitter tastant, and a bitterness inhibitor. Preferably, the composition includes an amount of bitterness inhibitor effective to block bitter taste perception arising from the bitter tastant.

In another aspect, the present invention provides a method of inhibiting a bitter taste of a reactive dental composition that results from contacting a taste tissue of a subject with a bitter tastant. The method includes: contacting the taste tissue with a treatment composition (e.g., a rinse solution or a rinse spray) that includes a bitterness inhibitor; and introducing the dental composition into the oral cavity of the patient. In some embodiments, the treatment composition (e.g., the rinse solution or the rinse spray) may be provided in a kit that also includes the reactive dental composition.

Other features and advantages of the present invention will be apparent from the following detailed description thereof, and from the claims.

### DETAILED DESCRIPTION

The present invention provides a means for reducing or eliminating the bitter taste of dental compositions that contain a bitter tastant. A “tastant” is defined as a compound or material that triggers a taste sensation in the oral cavity of a subject. Compositions of the present invention include an agent capable of blocking the perception of bitterness (i.e., a “bitterness inhibitor” as used herein, also known as a “bitter blocker” or a “bitterness blocker”), for example, by blocking the bitter taste receptors of the taste tissue of the patient. Preferably, the composition includes an amount of bitterness inhibitor effective to block bitter taste perception arising from a bitter tastant present, for example, in a dental composition.

“Effective amount,” as used herein, means that the bitterness inhibitor is present at a concentration that inhibits bitter taste perception.

For one example, a dental composition that includes an effective amount of bitterness inhibitor typically includes at least 0.01% by weight, preferably at least 0.05% by weight, and more preferably at least 0.1% by weight bitterness inhibitor, based on the total weight of the dental composition. In such an example, a dental composition that includes an effective amount of bitterness inhibitor typically includes at most 20% by weight, preferably at most 10% by weight, and more preferably at most 5% by weight bitterness inhibitor, based on the total weight of the dental composition.

For another example, a rinse solution or rinse spray that includes an effective amount of bitterness inhibitor typically includes at least 0.01 mM, preferably at least 0.1 mM, and more preferably at least 1 mM bitterness inhibitor. In such an example, a rinse solution or rinse spray that includes an effective amount of bitterness inhibitor typically includes at most 50 mM, preferably at most 20 mM, and more preferably at most 5 mM bitterness inhibitor.

Bitterness Inhibitors

Suitable bitterness inhibitors include, for example, nucleotides such as those described in, for example, WO 00/38536 (Margolskee et al.); WO 02/096464A1 (McGregor et al.); U.S. 2002/0177576 (McGregor et al.); and U.S. Pat. No. 6,540,978 (Margolskee et al.). A class of naturally occurring compounds that can block the transduction of bitter taste by interrupting the process at several points is also described by Ming et. al. (Ding Ming et al., Blocking taste receptor activation of gustducin inhibits gustatory responses to bitter compounds, Proc. Natl. Acad. Sci., August, 1999, 9903-9908, vol. 96, USA). In one embodiment, the bitterness inhibitor is a monophosphate, such as adenosine monophosphate.

Exemplary bitterness inhibitors include, for example, nucleotides (i.e., phosphate esters of nucleosides or nucleoside derivatives, and salts thereof) (e.g., sodium salts, disodium salts, potassium salts, dipotassium salts, lithium salts, ammonium salts, diammonium salts, alkylammonium salts, tris salts, and combinations thereof), and/or hydrates thereof. Preferred nucleotides include, for example, phosphate esters of ribonucleosides (e.g., adenosine, guanosine, cytidine, and uridine). More preferred nucleotides include phosphate esters of adenosine and phosphate esters of uridine. Exemplary phosphate esters include monophosphate esters (e.g., cyclic or non-cyclic), diphosphate esters, and combinations thereof. Suitable nucleotide monophosphate esters include, for example, 3-monophosphate esters, 5-monophosphate esters, and 3′,5′-cyclic monophosphate esters.

Preferred bitterness inhibitors include, for example, adenosine 3′-monophosphate and salts and/or hydrates thereof, adenosine 5′-monophosphate and salts and/or hydrates thereof, adenosine 3′,5′-cyclic monophosphate and salts and/or hydrates thereof, uridine 3′-monophosphate and salts and/or hydrates thereof, uridine 5′-monophosphate and salts and/or hydrates thereof, uridine 3′,5′-cyclic monophosphate and salts and/or hydrates thereof, and combinations thereof.

Dental Compositions

A dental composition of the present invention may be any reactive composition for use in the mouth of a patient, including a polymerizable and/or hardenable dental composition. Typically, the dental composition of the invention is a dental or orthodontic adhesive, a dental or orthodontic cement, a dental filling material or an impression material that contains a bitter-tasting ingredient. Such dental compositions often come in contact with the taste sensory organs and thus benefit from the addition of bitterness inhibitors in the formulation provided they do not substantially interfere with the setting times or other characteristics of the compositions.

As used herein, a “dental adhesive” refers to a non-filled or a lightly filled dental composition (e.g., less than 40% by weight filler), which is typically used to adhere a curable dental material (e.g., a filling material) to a tooth surface.

As used herein, a “dental cement” refers to a highly filled dental composition (e.g., at least 40% by weight filler), which is typically used to adhere a pre-formed or pre-cured dental article (e.g., an inlay, an onlay, a crown, or the like) to a tooth surface.

As used herein, an “orthodontic cement” refers to a composition that is typically used as a pre-treatment on a dental structure (e.g., a tooth) to adhere an orthodontic appliance (e.g., a band) to the dental structure.

As used herein, an “orthodontic adhesive” refers to a highly filled composition (e.g., at least 40% by weight filler), which is typically used to adhere an orthodontic appliance (e.g., a bracket) to a dental structure (e.g., tooth) surface. Generally, the dental structure surface is pre-treated, e.g., by etching, priming, and/or applying an adhesive to enhance the adhesion of the orthodontic adhesive or orthodontic cement to the dental structure surface.

As used herein, “impression material” refers to a material that is used in a softened or low viscosity form (uncured state) to make an accurate impression of hard and/or soft tissues within the oral cavity, and then cured to a hard or high viscosity form (cured state) that represents a negative model of the hard and/or soft tissues. In the cured state, the impression material needs to be able to receive a low viscosity material (e.g., a gypsum slurry) which after setting (i.e., hardening) represents a positive model of the hard and/or soft tissues of the mouth. The impression material is typically based on polyether or polysiloxane chemistry. Exemplary impression materials to which a bitterness inhibitor may be added include polyether impression materials, such as those described in, for example, U.S. Pat. No. 6,127,449 (Bissinger et al.); U.S. Pat. No. 6,395,801 (Bissinger et al.); and U.S. Pat. No. 5,569,691 (Guggenberger et al.).

As used herein, a “filling material” refers to a composition that is used to fill a defect in the tooth to restore its functionality. Often such filling materials are two part systems that cure gradually when these parts are mixed. Such materials could be glass ionomers, resin modified glass ionomers or self-curing resin-based composites typically with methacrylates or epoxy matrices.

The hardenable dental compositions of the present invention typically include a hardenable (e.g., polymerizable) component, thereby forming hardenable (e.g., polymerizable) compositions. In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically polymerizable, i.e., the compositions contain a chemical initiator (i.e., initiator system) that can polymerize, cure, or otherwise harden the composition without dependence on irradiation with actinic radiation. Such chemically polymerizable compositions are sometimes referred to as “self-cure” compositions and may include glass ionomer cements (e.g., conventional and resin-modified glass ionomer cements), redox cure systems, and combinations thereof.

Suitable photopolymerizable compositions may include epoxy resins (which contain cationically active epoxy groups), vinyl ether resins (which contain cationically active vinyl ether groups), ethylenically unsaturated compounds (which contain free radically active unsaturated groups), and combinations thereof. Examples of useful ethylenically unsaturated compounds include acrylic acid esters, methacrylic acid esters, hydroxy-functional acrylic acid esters, hydroxy-functional methacrylic acid esters, and combinations thereof. Also suitable are polymerizable materials that contain both a cationically active functional group and a free radically active functional group in a single compound. Examples include epoxy-functional acrylates, epoxy-functional methacrylates, and combinations thereof.

Photopolymerizable compositions may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include (meth)acrylates (i.e., acrylates and methacrylates) and (meth)acrylamides (i.e., acrylamides and methacrylamides), for example.

Fillers

The compositions of the present invention can also contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 0.1 micrometers, and more preferably less than 0.075 micrometer.

The filler can be an inorganic material. It can also be a crosslinked organic material that is insoluble in the resin system, and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz; nitrides (e.g., silicon nitride); glasses derived from, for example, Zr, Sr, Ce, Sb, Sn, Ba, Zn, and Al; feldspar; borosilicate glass; kaolin; talc; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz, submicron silica, and non-vitreous microparticles of the type described in U.S. Pat. No. 4,503,169 (Randklev). Mixtures of these non-acid-reactive fillers are also contemplated, as well as combination fillers made from organic and inorganic materials. Silane-treated zirconia-silica (Zr—Si) filler is especially preferred in certain embodiments.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than about 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like.

Other suitable fillers are disclosed in U.S. Pat. Nos. 6,387,981 (Zhang et al.) and 6,572,693 (Wu et al.) as well as International Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Nanofillers are also described in U.S. patent application Ser. Nos. 10/847,781; 10/847,782; and 10/847,803; all three of which were filed on May 17, 2004.

For some embodiments of the present invention that include filler (e.g., dental adhesive compositions), the compositions preferably include at least 1% by weight, more preferably at least 2% by weight, and most preferably at least 5% by weight filler, based on the total weight of the composition. For such embodiments, compositions of the present invention preferably include at most 40% by weight, more preferably at most 20% by weight, and most preferably at most 15% by weight filler, based on the total weight of the composition.

For other embodiments (e.g., wherein the composition is a dental restorative or an orthodontic adhesive), compositions of the present invention preferably include at least 40% by weight, more preferably at least 45% by weight, and most preferably at least 50% by weight filler, based on the total weight of the composition. For such embodiments, compositions of the present invention preferably include at most 90% by weight, more preferably at most 80% by weight, even more preferably at most 70% by weight filler, and most preferably at most 50% by weight filler, based on the total weight of the composition.

Glass Ionomers

The chemically polymerizable compositions may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. Nos. 5,063,257 (Akahane et al.), 5,520,725 (Kato et al.), 5,859,089 (Qian), 5,925,715 (Mitra) and 5,962,550 (Akahane et al.). In another type of RMGI, the cement may include a polycarboxylic acid, an acrylate or methacrylate-functional monomer, and a redox or other chemical cure system, e.g., as described in U.S. Pat. Nos. 5,154,762 (Mitra et al.), 5,520,725 (Kato et al.), and 5,871,360 (Kato). In another type of RMGI, the cement may include various monomer-containing or resin-containing components as described in U.S. Pat. Nos. 4,872,936 (Engelbrecht), 5,227,413 (Mitra), 5,367,002 (Huang et al.), and 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. patent application No. 2003/0087986 (Mitra).

Methods

A polymerizable (i.e., hardenable) dental composition of the present invention can be prepared by combining at least one nucleotide component, or other bitterness inhibitor, with one or more components of the composition using conventional mixing techniques. The resulting composition may optionally contain one or more dental additive as described herein.

The polymerizable compositions of the present invention can be supplied in a variety of forms including one-part systems and multi-part systems, e.g., two-part powder/liquid, paste/liquid, and paste/paste systems. Other forms employing multi-part combinations (i.e., combinations of two or more parts), each of which is in the form of a powder, liquid, gel, or paste are also possible. In a redox multi-part system, one part typically contains the oxidizing agent and another part typically contains the reducing agent.

The components of the hardenable composition can be included in a kit, where the contents of the composition are packaged to allow for storage of the components until they are needed.

When used as a dental composition, the components of the hardenable compositions can be mixed and clinically applied using conventional techniques. A curing light is generally required for the initiation of photopolymerizable compositions. The compositions can be in the form of composites or restoratives that adhere very well to dental structures. Optionally, a primer layer can be used on the dental structure on which the hardenable composition is used. The compositions, e.g., containing a FAS glass or other fluoride releasing material, can also provide very good long-term fluoride release. Some embodiments of the invention may provide resin modified glass ionomer cements or adhesives that can be cured in bulk without the application of light or other external curing energy, do not require a pre-treatment, have improved physical properties including improved flexural strength, and have high fluoride release for cariostatic effect.

In certain embodiments, the compositions of the present invention can include one or more dental additives. Exemplary dental additives include fluoride sources, whitening agents, anticaries agents (e.g., xylitol), remineralizing agents (e.g., calcium phosphate compounds), enzymes, breath fresheners, anesthetics, clotting agents, acid neutralizers, chemotherapeutic agents, immune response modifiers, medicaments, indicators, dyes, pigments, wetting agents, surfactants, buffering agents, viscosity modifiers, thixotropes, fillers, polyols, antimicrobial agents, antifungal agents, stabilizers, agents for treating xerostomia, desensitizers, and combinations thereof.

The dental compositions of the present invention can be useful as primers (including self-etching primers), adhesives (including self-etching adhesives), orthodontic adhesives, coatings, sealants, cements, and restoratives (including fillings, composites, flowables, and prostheses such as crowns, bridges, veneers, inlays, onlays, and the like). Dental prostheses typically are filled composites that are shaped and polymerized for final use before being disposed adjacent to a tooth. Such preformed articles can be ground or otherwise formed into a custom-fitted shape by the dentist or other user. When used as primers, adhesives, or cements, the dental compositions can be utilized for adhering a restorative (cured or uncured) to a dental structure surface. When used as an orthodontic adhesive, the dental composition can be utilized for adhering an orthodontic appliance (e.g., a bracket, a buccal tube, a band, a cleat, a button, a lingual retainer, or a bite blocker) to a dental structure surface.

Bitterness inhibitors may also be provided, for example, in the form of a rinse solution or a rinse spray to be used in conjunction with a dental composition as described herein. Preparation and application of solutions and sprays for the oral cavity are well known as described, for example, in U.S. Pat. Nos. 5,078,129 (Kleinberg et al.), 6,312,666 (Oxman et al.), 6,312,667 (Trom et al.), 6,540,978 (Margolskee et al.), 6,620,405 (Oxman et al.), and 6,669,927 (Trom et al.), and U.S. patent application Publication Nos. 2004/151691 (Oxman et al.), 2004/162375 (Ali et al.), and 2004/185013 (Burgio et al.). Typically, the rinse solution or the rinse spray is an aqueous solution.

For example, rinse solutions or rinse sprays can be applied to soft and/or hard tissues of the oral environment. Soft tissues include, for example, mucosal and gingival tissues. Hard tissues include, for example, teeth and their component parts (e.g., enamel, dentin, and cementum).

Rinse sprays as disclosed herein may be applied to the oral cavity as a fine mist or aerosol by any suitable means known in the art (e.g., U.S. Pat. Nos. 5,078,129 (Kleinberg et al.) and 6,620,405 (Oxman et al.)). For example, the rinse spray may be placed in a spray bottle and delivered with a hand pump. Alternatively, the rinse spray may be placed in a container with a propellant (e.g., air, nitrogen, carbon dioxide, and hydrocarbons) and be delivered using a pressurized spray can. In either case, the composition is passed through a fine orifice to form the fine mist.

A rinse solution or a rinse spray can be useful, for example, for pre-treating the oral cavity of a patient with a bitterness inhibitor to provide a method of reducing the bitter taste of a dental composition. A rinse solution or a rinse spray that includes a bitterness inhibitor may be provided to the practitioner in a kit that includes, in addition to the rinse solution or the rinse spray, a dental hardenable composition as described herein.

The following examples are provided to more particularly illustrate various embodiments of the present invention, and is in no way intended to be limiting thereof.

### EXAMPLES

### Examples 1-3 and Comparative Example 1 (CE-1)

Dental Cement Containing Bitterness Inhibitors

In order to demonstrate that nucleotide monophosphate additives do not adversely affect properties of two-part dental cement systems, the set times of a typical powder/liquid cement system were determined when a nucleotide monophosphate that can function as a bitterness inhibitor was added to a cement powder.

3M Unitek Multicure Band Cement (3M Unitek, Monrovia, Calif.) powder was mixed with the nucleotide monophosphates listed in Table I to provide powders A-C. The nucleotide monophosphates were obtained from Sigma-Aldrich, St. Louis, Mo. Powder D was the control and contained no additive. Each of the powders (1.9 parts by weight) was mixed with 1 part of 3M Unitek Multicure Band Cement liquid and the mixture spatulated for 30 seconds. The set times were determined at 37° C. and were reported from start of mixing. Three replicates were done for each cement mix and the results are reported in Table 1 as an average of the three replicates.

TABLE 1
Set Times of a Two-Part Cement System Having Varying
Concentrations of Different Nucleotide Monophosphate Additives

| | | | % By | Set Time |
|---|---|---|---|---|
| Example | Powder | Monophosphate | Weight | (Min:Sec) |
| 1 | A | Uridine 5′-Monophosphate | 0.51 | 4:00-4:10 |
| 2 | B | Adenosine 3′,5′-cyclic | 0.60 | 3:50-4:10 |
| | | Monophosphate | | |
| 3 | C | Adenosine 3′,5′-cyclic | 1.1 | 3:50-4:00 |
| | | Monophosphate Sodium Salt | | |
| CE-1 | D | None (Control) | 0 | 3:50-4:10 |

As shown by the data presented in Table 1, the addition of the monophosphates did not adversely affect the setting of the cement. Cement samples from Examples 2 and 3 had improved tastes over the control Example CE-1. The cement from Example 1 was not tested for taste.

### Examples 4-5 and Comparative Example 2 (CE-2)

Dental Restorative System Containing a Bitterness Inhibitor

In order to demonstrate that nucleotide monophosphate additives do not adversely affect properties of two-part dental restorative systems, the set times of a typical glass ionomer restorative system were determined when a nucleotide monophosphate that can function as a bitterness inhibitor was added to a restorative powder.

3M ESPE Vitremer Tri-cure Glass Ionomer System (a glass ionomer filling material from 3M ESPE, St. Paul, Minn.) powder was mixed with the nucleotide adenosine 5′-monophosphate sodium salt (Linguagen Corp., Cranbury, N.J.) to provide powders E and F. Powder G was the control and contained no additive. The concentrations of monophosphate in the glass ionomer powder are shown in Table 2. Each of the powders (2.5 parts by weight) was mixed with 1 part of 3M ESPE Vitremer Tri-cure Glass Ionomer System liquid and spatulated for 30 seconds. The set times were determined at 37° C. and were reported from start of mixing. The light-curing behavior of the restorative systems as well as that of the control were examined in order to determine if the nucleotide monophosphate affected this property.

A fresh mix of each of the restorative materials (i.e., cements) was cured in a Teflon mold 5-mm in diameter and 5-mm in height. The materials in the molds were irradiated at 24° C. with a Freelight 2 Dental Curing Light (3M ESPE). The undersides of the cements (away from the light source) were scraped back to remove uncured material and the depth of cured cement determined using calipers. Three replicates were done for each cement mix and the average results reported in Table 2.

TABLE 2
Set Times and Cure Depths of a Two-Part Glass Ionomer Restorative
System Having Varying Concentrations of Adenosine
5′-Monophosphate Sodium Salt Additive

| | | % By Weight | Set Time | |
|---|---|---|---|---|
| Example | Powder | Monophosphate | (Min:Sec) | Depth of Cure |
| 4 | E | 1.0 | 3:50-4:00 | 2.5 mm |
| 5 | F | 2.0 | 4:00-4:10 | 2.5 mm |
| CE-2 | G | 0 | 3:50-4:00 | 2.5 mm |

As shown by the data presented in Table 2, the addition of the monophosphate did not adversely affect the dark-cure or light-cure setting behaviors of the glass ionomer restorative. Cement samples from Examples 4 and 5 had improved tastes over the control Example CE-2.

### Examples 6-7 and Comparative Example 3 (CE-3)

Dental Impression Material Containing a Bitterness Inhibitor

In order to demonstrate that nucleotide monophosphate additives do not adversely affect properties of dental impression materials, the start-of-setting times of a typical impression material were determined when a nucleotide monophosphate that can function as a bitterness inhibitor was added to the catalyst paste of the material.

3M ESPE impression material Impregum Penta Soft medium body (3M ESPE, Seefeld, Germany) catalyst paste was mixed with the nucleotide adenosine 5-monophosphate sodium salt (Linguagen Corp., Cranbury, N.J.) using a speed mixer to provide catalyst pastes A and B. Paste C was the control and contained no additive. The concentrations of monophosphate in the catalyst pastes are shown in Table 3. Each of the catalyst pastes (0.5 g) was mixed on a mixing pad with 0.12 g of base paste and spatulated without voids and bubbles for 45 seconds. Approximately half of the amounts of the mixed pastes were smoothed out on the mixing pad. The remaining amounts were used to determine the start-of-setting times.

The start-of-setting times were determined at 23° C. and were reported from start of mixing. After mixing, the pastes remained stringy and flowable until a sharp transition point was reached where the pastes could no longer flow and be shaped. The time from start of mix until this transition point was reached was defined as the start-of-setting time. Three replicates were done for each impression material and the results are reported in Table 3 as an average of the three replicates.

TABLE 3
Start-of Setting Times of a Two-Part Polyether Impression Material
Having Varying Concentrations of Adenosine
5′-Monophosphate Sodium Salt Additive.

| | | % By Weight | Start-of-Setting Time |
|---|---|---|---|
| Example | Catalyst Paste | Monophosphate | (Min:Sec) |
| 6 | A | 0.25 | 2:10-2:15 |
| 7 | B | 2.0 | 2:10-2:15 |
| CE-3 | C | 0 | 2:10-2:15 |

As shown by the data presented in Table 3, the addition of the monophosphate did not adversely affect the start-of-setting time of the impression material.

The present invention has been described with reference to several embodiments thereof. The foregoing description of specific embodiments and examples has been provided to illustrate the invention, and is not intended to be limiting of the scope of the invention. It will be apparent to those skilled in the art that many changes can be made to the described embodiments without departing from the spirit and scope of the invention.

All patents, applications, and publications mentioned above are incorporated by reference herein.

1. A dental composition comprising a reactive component, a bitter tastant, and a bitterness inhibitor, wherein the bitterness inhibitor comprises a nucleotide or hydrate thereof, a nucleotide salt or hydrate thereof, or a combination thereof.

2. The dental composition of claim 1, wherein the reactive component comprises a hardenable or polymerizable component.

3. The dental composition of claim 1, wherein the composition is a dental adhesive, an orthodontic adhesive, a dental cement, an orthodontic cement, or a filling material.

4. The dental composition of claim 3 wherein the orthodontic cement is an orthodontic band cement.

5. The dental composition of claim 1, wherein the composition is an impression material.

6. The dental composition of claim 5, wherein the impression material comprises a polyether.

7. (canceled)

8. The dental composition of claim 1, wherein the nucleotide or nucleotide salt is a monophosphate ester.

9. The dental composition of claim 8, wherein the bitterness inhibitor comprises an adenosine monophosphate or a uridine monophosphate.

10. The dental composition of claim 1, wherein the composition comprises an amount of bitterness inhibitor effective to block bitter taste perception arising from the bitter tastant.

11. A method of inhibiting a bitter taste of a reactive dental composition resulting from contacting a taste tissue of a subject with a bitter tastant, the method comprising administering to the subject a composition according to claim 1.

12. A method of inhibiting a bitter taste of a reactive dental composition resulting from contacting a taste tissue of a subject with a bitter tastant, the method comprising:
contacting the taste tissue with a treatment composition comprising a bitterness inhibitor comprising a nucleotide hydrate thereof or nucleotide salt or hydrate thereof, or a combination thereof; and
introducing the dental composition into the oral cavity of the patient.

13. The method of claim 12, wherein the treatment composition comprises a rinse solution or a rinse spray.

14. The method of claim 12 wherein the reactive dental composition comprises a reactive component and a bitter tastant.

15. The method of claim 14, wherein the reactive component comprises a hardenable or polymerizable component.

16. The method of claim 12, wherein the dental composition is a dental adhesive, an orthodontic adhesive, a dental cement, an orthodontic cement, or a filling material.

17. The method of claim 16 wherein the orthodontic cement is an orthodontic band cement.

18. The method of claim 12, wherein the dental composition is an impression material.

19. The method of claim 18 wherein the impression material comprises a polyether.

20. (canceled)

21. The method of claim 12 20, wherein the nucleotide or nucleotide salt is a monophosphate ester.

22. The method of claim 21, wherein the bitterness inhibitor comprises an adenosine monophosphate or a uridine monophosphate.

23. A kit comprising:
a reactive dental composition; and
a rinse solution comprising a bitterness inhibitor, comprising a nucleotide or hydrate thereof, or nucleotide salt or hydrate thereof, or a combination thereof.

24. A kit comprising:
a reactive dental composition; and
a rinse spray comprising a bitterness inhibitor, comprising a nucleotide or hydrate thereof, or nucleotide salt or hydrate thereof, or a combination thereof.
